(19) United States
(12) Patent Application Publication
(10) Pub. No.: US 2023/0008738 A1
(43) Pub. Date: Jan. 12, 2023

(54) THREE-WAY UROLOGY CATHETER STOPCOCK AND UROLOGY CATHETER STOPCOCK ASSEMBLY

(71) Applicant: The University of Toledo, Toledo, OH (US)

(72) Inventors: Sheila Rothert, Toledo, OH (US); Cindy Peters, Toledo, OH (US); Rebecca Duvall, Toledo, OH (US); Laura Shoup, Toledo, OH (US); Janelle Tipton, Toledo, OH (US); Eric Betka, Toledo, OH (US); David Otte, Toledo, OH (US); Marc Crisenbery, Toledo, OH (US)

(73) Assignee: The University of Toledo, Toledo, OH (US)

(21) Appl. No.: 17/859,260

(22) Filed: Jul. 7, 2022

Related U.S. Application Data
(60) Provisional application No. 63/218,975, filed on Jul. 7, 2021.

(51) Int. Cl. A61M 39/22; A61M 39/10; A61M 39/20

(52) U.S. Cl. CPC A61M 39/223; A61M 39/20; A61M 39/105; A61M 2039/229

(57) ABSTRACT

A three-way urology catheter stopcock includes a stopcock body defining a longitudinally extending fluid flow passage, the body having three connector mounting openings in a wall thereof, an on/off selector movably mounted within the fluid flow passage of the body, the on/off selector including a longitudinally extending, cylindrical selector body having an open end, a closed end, and three fluid flow openings in a wall of the selector body, wherein the closed second end includes one of a plurality of fluid flow indicator members and fluid flow indicia, two first connectors mounted in opposing connector mounting openings of the stopcock body, and a second connector mounted in the connector mounting opening that is between the openings in which the two first connectors are mounted.

### BACKGROUND OF THE INVENTION

This invention relates in general to urology catheters. In particular, this invention relates to a urology catheter with an improved structure designed to maintain sterility for patient safety and to protect medical personnel, such as nurses, from hazardous spills.

According to the CDC, each year in the United States approximately 85,000 people are diagnosed with bladder cancer. About 70 percent of those diagnosed are caught at an early stage, i.e., a non-muscle invasive disease, with cancer limited to the epithelium, the tissue lining the bladder. Treatment for this early stage bladder cancer varies, but often involves weekly bladder instillations for six weeks, and then three monthly treatments with a follow-up cystoscopy, and then if the cancer is determined to be stable, the three monthly treatments and cystoscopy may be repeated for 1-3 years.

Additionally, with known bagged catheters, it may not be possible to introduce drugs into the bladder because the bag prevents access to the end of the catheter. One known method of overcoming this limitation and administering cancer medications to a patient’s bladder introduces new challenges to the process and includes placing a clamp on the catheter to prevent the nurse or clinician from being splashed with urine, and removing the red tape that defines a sterile seal. The nurse then connects a medication syringe to the catheter opening, releases the clamp, and administers the medication while being careful to contain urine and chemotherapy/biotherapy agent spills with gauze and barriers. The catheter is then either removed if the patient is deemed able to hold the medicine in the bladder for 1 to 2 hours and then void the bladder in a toilet, or the catheter is then clamped again to allow the medication to dwell for 1 to 2 hours, after which the catheter is unclamped, the urine and medicine are drained, and the catheter is removed. When more than one medication must be administered, this process may also be performed repeatedly.

Removing the red tape from the urology catheter and manipulation the urology catheter so close to the patient many times during a procedure is also not conducive to patient comfort and privacy. Such frequent catheter manipulation also can cause trauma to the bladder or the urethra, thus making the patient more susceptible to infection.

The United States Pharmacopeia (USP) Convention has published standards to protect healthcare workers from hazardous drugs used in the intravesicular treatment for early bladder cancer, such as, but not limited to, mitomycin, BCG, gemcitabine, and docetaxel.

Thus, it would be desirable to provide an improved structure for a urology catheter that allows for intravesicular medication delivery to the bladder, containing the medication in the bladder for a specified period of time, and draining urine and medication from the bladder into a urine drainage bag while maintaining a sterile environment and preventing spills of urine and hazardous chemotherapy/biotherapy agents.

### SUMMARY OF THE INVENTION

This invention relates to an improved structure for a three-way urology catheter stopcock. When used in combination with a urology catheter, the three-way urology catheter stopcock allows for intravesicular medication delivery to the bladder, containing the medication in the bladder for a specified period of time, and draining urine and medication from the bladder into a urine drainage bag while maintaining a sterile environment and preventing spills of urine and hazardous chemotherapy/biotherapy agents. The three-way urology catheter stopcock includes a stopcock body defining a longitudinally extending fluid flow passage, the body having three connector mounting openings in a wall thereof, an on/off selector movably mounted within the fluid flow passage of the body, the on/off selector including a longitudinally extending, cylindrical selector body having an open end, a closed end, and three fluid flow openings in a wall of the selector body, wherein the closed second end includes one of a plurality of fluid flow indicator members and fluid flow indicia, two first connectors mounted in opposing connector mounting openings of the stopcock body, and a second connector mounted in the connector mounting opening that is between the openings in which the two first connectors are mounted.

Various aspects of this invention will become apparent to those skilled in the art from the following detailed description of the preferred embodiment, when read in light of the accompanying drawings.

### BRIEF DESCRIPTION OF THE DRAWINGS

FIG. 1 is a perspective view of the three-way urology catheter stopcock according to this invention.

FIG. 2 is a partially exploded perspective view of the three-way urology catheter stopcock illustrated in FIG. 1.

FIG. 3 is an exploded perspective view of the three-way urology catheter stopcock illustrated in FIGS. 1 and 2.

FIG. 4 is a top plan view of the three-way urology catheter stopcock illustrated in FIGS. 1 through 3 and showing the stopcock in a first position.

FIG. 5 is a top plan view of the three-way urology catheter stopcock illustrated in FIGS. 1 through 4 and showing the stopcock in a second position.

FIG. 6 is a top plan view of the three-way urology catheter stopcock illustrated in FIGS. 1 through 5 and showing the stopcock in a third position.

FIG. 7 is a top plan view of the three-way urology catheter stopcock illustrated in FIGS. 1 through 6 and showing the stopcock in a fourth position.

FIG. 8 is a perspective view of a conventional urology catheter.

### DETAILED DESCRIPTION OF THE PREFERRED EMBODIMENT

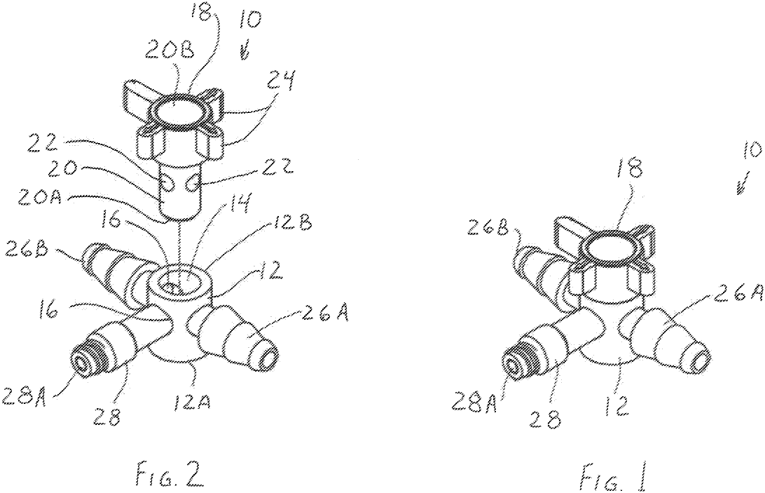

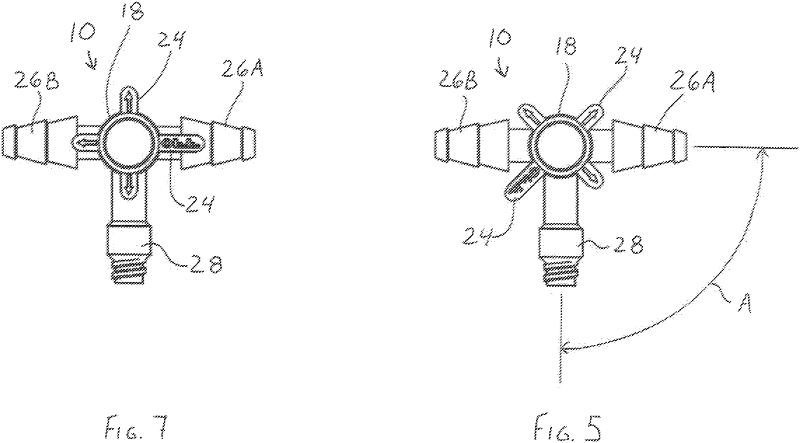

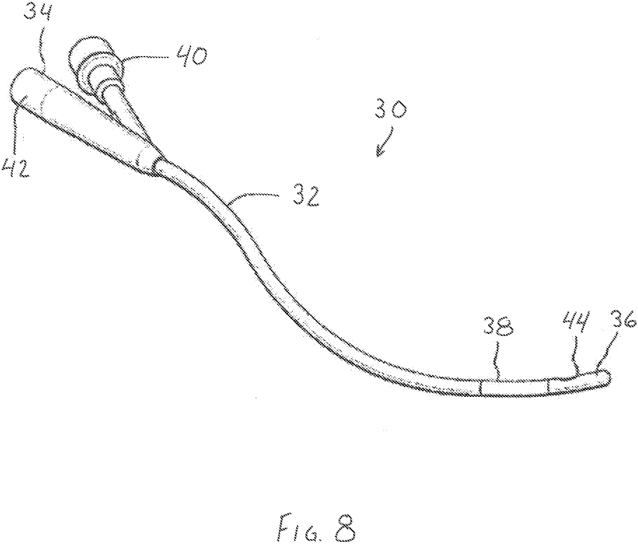

Referring now to the drawings, there is illustrated at 10 in FIGS. 1 through 7 an embodiment of a three-way urology catheter stopcock according to this invention.

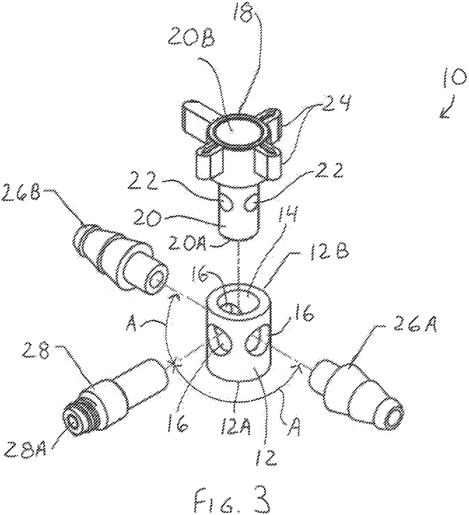

The three-way urology catheter stopcock 10 includes a generally cylindrical stopcock body 12 defining a longitudinally extending fluid flow passage 14, a first end 12A (the lower end when viewing FIGS. 1 through 3) and a second end 12B (the upper end when viewing FIGS. 1 through 3). The body 12 has three connector mounting openings 16 in a wall of the stopcock body 12. The three connector mounting openings 16 are positioned around the wall of the stopcock body 12 at 90 degree intervals (see angle A in FIGS. 3 and 5).

An on/off selector 18 is movably mounted within the fluid flow passage 14 of the body 12. The selector 18 includes a longitudinally extending, generally cylindrical selector body 20, a first, open end 20A (the lower end when viewing FIGS. 1 through 3) and a second, closed end 20B (the upper end when viewing FIGS. 1 through 3). The selector 18 has three fluid flow openings 22 in a wall of the selector body 20. Like the three connector mounting openings 16, the three fluid flow openings 22 are positioned around the wall of the selector body 20 at 90 degree intervals.

The closed second end 20B includes fluid flow indicator members 24 and/or fluid flow indicia. For example, in the illustrated embodiment, fluid flow directional arrows and the word “off” are provided.

The three-way urology catheter stopcock 10 further includes two first connectors 26A and 26B mounted in opposing connector mounting openings 16 of the stopcock body 12. In the illustrated embodiment, the first connectors 26A and 26B are barbed fittings configured for push-on connection to a flexible hose or tube, such as a conventional urology catheter 30, described below, and a drain tube 46 of a conventional urology drainage bag (not shown), also described below. Thus, the first connector 26A defines a catheter port, and the first connector 26B defines a drainage bag port. Red tape may be used to securely connect the first connectors 26A and 26B to Foley catheter tubing and drainage bag tubing. In some embodiments of using the three-way urology catheter stopcock 10, the red tape would only be removed in case of medical necessity, such as a patient emergency, or when there is a defect in the tubing or other materials.

The three-way urology catheter stopcock 10 also includes a second or Luer connector 28 mounted in the connector mounting opening 16 that is between the openings 16 in which the first connectors 26A and 26B are mounted. In the illustrated embodiment, the Luer connector 28 is a normally closed needle-free fitting with a male Luer thread configured for connection to a corresponding female Luer fitting 29 on a conventional medication dispensing device, such as a syringe (not shown). Thus, the Luer connector 28 defines a medication dispensing port 28A. The medication dispensing port 28A may include a sterile cap, such as schematically shown at 31 in FIG. 6. The sterile cap 31 may be attached to the medication dispensing port 28A via threaded connection and function as a cover to protect the medication dispensing port 28A from potential contamination. One example of such a sterile cap 31 is the SwabCap® manufactured by B. Braun Medical Inc. of Bethlehem, PA.

The three-way urology catheter stopcock 10 may be formed from plastic, such as polypropylene, polytetrafluoroethylene (PTFE), high-density polyethylene (HDPE), acrylonitrile butadiene styrene (ABS), polylactic acid (PLA), or any desired material.

One embodiment of a conventional urology catheter is shown at 30 in FIG. 8. The catheter 30 includes a catheter body 32 having a proximal end 34 and a distal end 36. The catheter 30 also includes a balloon 38, an inflation lumen 40, and a drainage lumen 42. The balloon 38 is disposed near the distal end 36 and is deflated for insertion into a patient. The inflation lumen 40 extends within the catheter body 32 from the proximal end 34 to the balloon 38, is in fluid communication with the balloon 38 for inflating and deflating the balloon 38. The catheter drainage lumen 42 extends from the proximal end 34 to the distal end 36. The distal end 36 includes an opening 44 in fluid communication with the drainage lumen 42 to facilitate drainage of urine from the bladder of the patient.

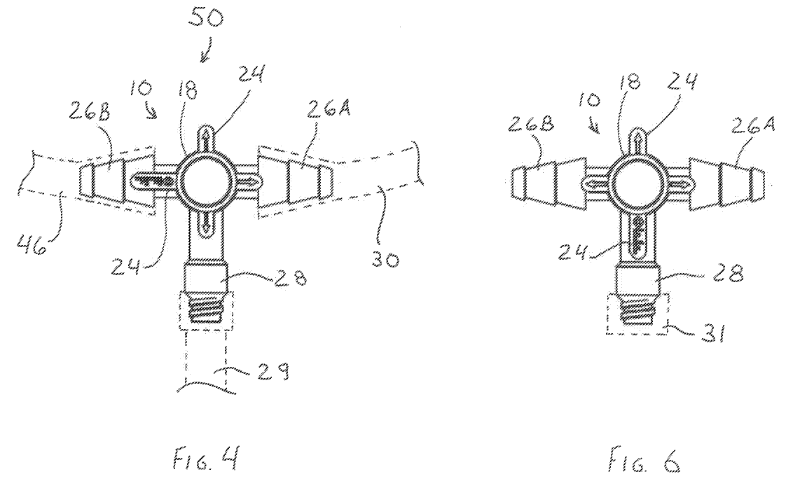

As best shown in FIG. 4, the catheter port 26A is configured for attachment to the drainage lumen 42 at the proximal end 34 of the catheter 30, the drainage bag port 26B is configured for attachment to the drainage tube 46 of a conventional urology drainage bag (not shown), and the medication dispensing port 28A is configured for connection to the corresponding female Luer fitting 29 on the medication dispensing device, such as a syringe (not shown).

It will be understood that the three-way urology catheter stopcock 10 may be assembled with one or both of the catheter 30, and the drainage bag (not shown) to define a three-way urology catheter stopcock assembly 50. The three-way urology catheter stopcock 10, whether sold alone or included as part of a kit that also includes one or both of the catheter 30 and the drainage bag, and is thus configured to form the three-way urology catheter stopcock assembly 50, may include the sterile cap 31 on the Luer fitting that defines the medication dispensing port 28A.

In use, the three-way urology catheter stopcock 10 may be moved to a desired position by rotating the on/off selector 18 such that two of the fluid flow openings 22 in the wall of the selector body 20 are aligned with the desired two of the stopcock ports 26A, 26B, and 28 in the stopcock body 12, as shown in FIGS. 4, 6, and 7.

Additionally, the on/off selector 18 may be rotated such that none of the fluid flow openings 22 in the wall of the selector body 20 are aligned with any of the stopcock ports 26A, 26B, and 28 in the stopcock body 12, as shown in FIG. 5.

Specifically, the three-way urology catheter stopcock 10 is moveable between four positions: a first or delivery stopcock position is shown in FIG. 4 wherein a fluid flow path is open between the medication dispensing device (see the fitting 29 in FIG. 4) and the catheter 30, and the drainage bag port 26B is closed; a second or dwell stopcock position is shown in FIG. 5 wherein each of the medication dispensing port 28A, the catheter port 26A, and the drainage bag port 26B are closed; a third or drain stopcock position is shown in FIG. 6 wherein a fluid flow path is open between the catheter 30 and the drainage bag (via the drainage tube 46), and the medication dispensing port 28A is closed; and if desired, the three-way urology catheter stopcock 10 may be moved to a position a fourth position wherein a flow path is open between the medication dispensing device (see the fitting 29 in FIG. 4) and the drainage bag (via the drainage tube 46), and the catheter port 26A is closed. Thus, the three-way urology catheter stopcock 10 may be referred to as a deliver/dwell/drain urology catheter stopcock.

The three-way urology catheter stopcock 10 may be provided in any desired number of commonly used channel sizes designed for intravesicular medication delivery to the bladder. In use, the three-way urology catheter stopcock 10 may be moved to the delivery stopcock position is shown in FIG. 4 for intravesicular medication delivery to the bladder. Once a desired amount of medication has been delivered to the bladder, the three-way urology catheter stopcock 10 may then be rotated to the dwell stopcock position shown in FIG. 5 to contain the medication in the bladder for a period of time determined by the medication delivered, such as for example between 1 and 2 hours. At the completion of the medication dwell time, the three-way urology catheter stopcock 10 may then be rotated to the drain stopcock position shown in FIG. 6 to allow urine and the medication in the bladder to drain into the drainage bag.

The Luer connector 28 has been described and illustrated as having a size and diameter compatible with standard, conventional Luer fittings. It is known that conventional Luer fittings are used to connect to intravenous devices for the administration of intravenous medication, and are also used to connect to intravesicular devices for the administration of medication, such as cytotoxic drugs used in chemotherapy to kill cancer cells.

Thus, in an alternate embodiment of the three-way urology catheter stopcock 10, the Luer connector 28 may be manufactured having a size, shape, and/or diameter larger than a conventional Luer connector, such as for example about 1 mm larger in diameter. Advantageously, if the Luer connector 28 is larger and thus incompatible with conventional Luer fittings, it would not be possible for a user, such as a medical professional or a patient, to mistakenly connect an intravesicular medication dispensing device and administer an intravesicular medication via a Luer fitting on an intravenous line or device. Similarly, an intravenous medication dispensing device would not be compatible or attachable to an intravesicular device.

Advantageously, the three-way urology catheter stopcock 10 shown in FIGS. 1 through 7 protects caregivers and patients from hazardous spills, provides medical personnel a tool for meeting USP standards for intravesicular medication delivery, maintains an aseptic environment that prevents infections, and does not require the red tape (sterile seal) to be removed from the catheter 30. The three-way urology catheter stopcock 10 also has an easily accessible Luer port (medication dispensing port) which is also compatible with any closed system transfer device for hazardous medicine delivery described in USP standards, allows patient privacy to be maintained, results in less trauma to the bladder from catheter 30 manipulation during the delivery process, and is easy to use.

Additionally, the National Institute for Occupational Safety and Health (NIOSH) has recommended that healthcare workers should use an effective Closed System Transfer Device (CSTD) in order to minimize exposure to hazardous drugs and limit their adverse effects. An effective CSTD, as defined by NIOSH, is a system that “mechanically prohibits the transfer of environmental contaminants into the system and the escape of hazardous drug or vapor outside the system.” Advantageously, the three-way urology catheter stopcock 10 and the three-way urology catheter stopcock assembly 50 described herein provide such a closed system for the safe handling of hazardous drugs. Both the three-way urology catheter stopcock 10 and the three-way urology catheter stopcock assembly 50 have a unique design that is easy for clinicians to use and is discrete and comfortable for patients.

The principle and mode of operation of this invention have been explained and illustrated in its preferred embodiment. However, it must be understood that this invention may be practiced otherwise than as specifically explained and illustrated without departing from its spirit or scope.

What is claimed is:

1. A three-way urology catheter stopcock comprising:
a stopcock body defining a longitudinally extending fluid flow passage, the body having three connector mounting openings in a wall thereof;
an on/off selector movably mounted within the fluid flow passage of the body, the on/off selector including a longitudinally extending, cylindrical selector body having an open end, a closed end, and three fluid flow openings in a wall of the selector body;
wherein the closed second end includes one of a plurality of fluid flow indicator members and fluid flow indicia;
two first connectors mounted in opposing connector mounting openings of the stopcock body; and
a second connector mounted in the connector mounting opening that is between the openings in which the two first connectors are mounted.

2. The three-way urology catheter stopcock according to claim 1, wherein the stopcock body is cylindrical.

3. The three-way urology catheter stopcock according to claim 1, wherein the two first connectors define a catheter port and a drainage bag port.

4. The three-way urology catheter stopcock according to claim 1, wherein the two first connectors are barbed fittings configured for push-on connection to a flexible tube.

5. The three-way urology catheter stopcock according to claim 1, wherein the second connector is a normally closed needle-free fitting with a male Luer thread.

6. The three-way urology catheter stopcock according to claim 5, wherein the second connector is configured for connection to a corresponding female Luer fitting on a medication dispensing device.

7. The three-way urology catheter stopcock according to claim 1, wherein the second connector defines a medication dispensing port.

8. The three-way urology catheter stopcock according to claim 1, wherein the three connector mounting openings of the stopcock body are positioned around the wall of the stopcock body 12 at 90 degree intervals.

9. The three-way urology catheter stopcock according to claim 1, wherein the three fluid flow openings of the on/off selector are positioned around the wall of the selector body at 90 degree intervals.

10. A three-way urology catheter stopcock assembly comprising:
a three-way urology catheter stopcock; and
a urology catheter;
wherein the three-way urology catheter stopcock includes:
a stopcock body defining a longitudinally extending fluid flow passage, the body having three connector mounting openings in a wall thereof;
an on/off selector movably mounted within the fluid flow passage of the body, the on/off selector including a longitudinally extending, cylindrical selector body having an open end, a closed end, and three fluid flow openings in a wall of the selector body;
wherein the closed second end includes one of a plurality of fluid flow indicator members and fluid flow indicia;
two first connectors mounted in opposing connector mounting openings of the stopcock body; and
a second connector mounted in the connector mounting opening that is between the openings in which the two first connectors are mounted.

11. The three-way urology catheter stopcock assembly according to claim 10, further including a urology drainage bag.

12. The three-way urology catheter stopcock assembly according to claim 10, wherein the stopcock body is cylindrical.

13. The three-way urology catheter stopcock assembly according to claim 10, wherein the two first connectors define a catheter port and a drainage bag port.

14. The three-way urology catheter stopcock assembly according to claim 10, wherein the two first connectors are barbed fittings configured for push-on connection to a flexible tube.

15. The three-way urology catheter stopcock assembly according to claim 10, wherein the second connector is a normally closed needle-free fitting with a male Luer thread.

16. The three-way urology catheter stopcock assembly according to claim 15, wherein the second connector is configured for connection to a corresponding female Luer fitting on a medication dispensing device.

17. The three-way urology catheter stopcock assembly according to claim 10, wherein the second connector defines a medication dispensing port.

18. The three-way urology catheter stopcock assembly according to claim 10, wherein the three connector mounting openings of the stopcock body are positioned around the wall of the stopcock body 12 at 90 degree intervals.

19. The three-way urology catheter stopcock assembly according to claim 10, wherein the three fluid flow openings of the on/off selector are positioned around the wall of the selector body at 90 degree intervals.

20. The three-way urology catheter stopcock assembly according to claim 10, further comprising a sterile cap configured to be attached to the second connector via a threaded connection.

21. A three-way urology catheter stopcock assembly comprising:
a three-way urology catheter stopcock;
a urology drainage bag; and a urology catheter;
wherein the three-way urology catheter stopcock includes:
a stopcock body defining a longitudinally extending fluid flow passage, the body having three connector mounting openings in a wall thereof;
an on/off selector movably mounted within the fluid flow passage of the body, the on/off selector including a longitudinally extending, cylindrical selector body having an open end, a closed end, and three fluid flow openings in a wall of the selector body;
wherein the closed second end includes one of a plurality of fluid flow indicator members and fluid flow indicia;
two first connectors mounted in opposing connector mounting openings of the stopcock body, wherein the two first connectors are barbed fittings configured for push-on connection to a flexible tube; and
a second connector mounted in the connector mounting opening that is between the openings in which the two first connectors are mounted, wherein the second connector is a normally closed needle-free fitting with a male Luer thread.

22. The three-way urology catheter stopcock assembly according to claim 21, further comprising a sterile cap configured to be attached to the second connector via a threaded connection.
